(19) United States
(12) Patent Application Publication
(10) Pub. No.: US 2020/0071658 A1
(43) Pub. Date: Mar. 5, 2020

(54) METHOD AND APPARATUS FOR THE ISOLATION AND TREATMENT OF PARTICULATE TARGETS

(71) Applicant: Molecular Machines & Industries AG, Glattbrugg (CH)

(72) Inventors: Stefan SEEGER, Zollikon (CH); Stefan NIEHREN, Altomunster (DE)

(73) Assignee: Molecular Machines & Industries AG, Glattbrugg (CH)

(21) Appl. No.: 16/471,142

(22) PCT Filed: Dec. 20, 2017
(86) PCT No.: PCT/EP2017/083872

§ 371 Date: Jun. 19, 2019

(30) Foreign Application Priority Data
Dec. 30, 2016 (EP) 16207486.8

(51) Int. Cl. C12M 1/00; B01L 3/02; B01L 3/00; G01N 1/04; G01N 1/40

(52) U.S. Cl. CPC C12M 47/06; B01L 3/021; B01L 3/502761; C12M 47/04; G01N 1/04; G01N 21/64; B01L 2200/0668; B01L 2300/0838; B01L 2400/049; G01N 2001/045; G01N 1/4044

(57) ABSTRACT

A method of isolating/treating a particulate target using an apparatus comprising at least a capillary comprising a tip orifice and an internal cavity comprising a treatment liquid, a positioning element capable of positioning the tip orifice of the capillary, and a discharging/drawing element capable of causing a discharge of treatment liquid from the capillary and causing a drawing of treatment liquid into the capillary. The method comprises the steps of (1) locating the particulate target to be isolated and treated, (2) positioning the tip orifice such that the tip orifice comes in proximity to the particulate target, using the positioning element, (3) discharging a volume of treatment liquid to enclose the particulate target in the treatment liquid, (4) drawing the particulate target enclosed in the treatment liquid into the internal cavity of the capillary, and (5) treating the particulate target within the internal cavity of the capillary comprising the treatment liquid.

### TECHNICAL FIELD

The present invention relates to a method of isolating and treating a particulate target located on a substrate using a capillary cell sorting apparatus.

### STATE OF THE ART

Isolated cells from tumours, endothelial tissue, histocytes, stem cells, and other materials are a prerequisite for analysis and patient profiling in medical diagnostic applications or life science research. However, current methods of isolation of single particulate targets such as cells correspond more often to enrichment methods rather than a true isolation of single cells.

For instance, filters designed to isolate epithelial tumour cells such as circulating cancer cells (CTCs) by size from bodily fluids such as blood are commercialized by Rarecells SAS and in fact rather enrich such cells. This means that the plurality of cells trapped in the filters are neither identical with respect to the cell type nor genetically speaking. It is therefore not possible to analyse the collected cells in bulk and individual cells must therefore be individually isolated in order to determine their genotype.

It is further possible to isolate individual cells or cell clusters from a tissue sample by laser microdissection methods, of which multiple variations exist. For example, WO2006031574 A1 describes the use of a specific laser based technique, called laser capture microdissection, in which a small cell area can be extracted from biological tissue samples by means of an IR-laser induced transfer utilising a thermoplastic membrane. Another variation, described in EP 0 879 408 B1 consists in a technique called laser catapult technology. First a laser is used to cut around an area of interest such as a cell in a tissue sample and second the area is catapulted into a collection vessel by using laser pulses. DE 10003588 C1 discloses a laser microdissection technology in which in a first step a laser is used for excising cells from a tissue sample on a membrane slide and isolate the cell in a second step by adhering the cut area onto an adhering cap, and subsequently, a lysis buffer is used to extract cellular components like DNA or RNA for further genetic analysis.

All of the above methods require two discrete steps: A first step of selecting and isolating the target cell from its environment and/or substrate and a subsequent second step of transferring the target cell to a separate vessel where then a treatment liquid such as for example a lysis buffer is added to the previously isolated target cell. Such methods are labour intensive, even if automated, require the use of additional containers and materials such as transfer foils and testing tubes.

While the excision of the cells from tissue samples has been mastered using laser microdissection, such methods are unsuited for isolating adherent cells, in particular cultured cells, from the bottom of a cell culture container.

It is thus desirable to provide a method, as well as a suited apparatus, by which the single particulate targets, such as for example cells, can be isolated and treated in a more effective and simple manner such as to streamline the handling of such single particulate targets.

### SUMMARY OF THE INVENTION

The method and the apparatus of the present invention overcome the above problems by providing a method and apparatus in which the isolation and treatment of a target particulate such as a cell are carried out concurrently, by in essence drawing the particulate target into a capillary filled with a treatment liquid and carrying out the treatment inside the capillary instead of transferring the particulate target to a separate vessel for treatment.

It is thus an object of the present invention to provide a method of isolating and treating a particulate target, preferably located on a substrate, using an apparatus comprising
at least a capillary comprising a tip orifice and an internal cavity, said internal cavity comprising a treatment liquid,
a positioning element capable of positioning the tip orifice of the capillary,
a discharging/drawing element capable of causing a discharge of liquid from the capillary as well as causing a drawing of liquid into the capillary,
said method comprising the steps of:

a. locating on the substrate the particulate target to be isolated and treated;

b. positioning the tip orifice of the capillary such that the tip orifice of the capillary comes in proximity to the particulate target using the positioning element;

c. discharging a sufficient volume of treatment liquid, preferably from the internal cavity of the capillary, such as to essentially enclose the particulate target in the treatment liquid using the discharging/drawing element;

d. drawing the particulate target enclosed in the treatment liquid into the internal cavity of the capillary,

e. treating the particulate target within the internal cavity of the capillary comprising the treatment liquid.

It is further an object of the present invention to provide a method of isolating and lysing a target cell, preferably located on a substrate, using an apparatus comprising
at least a capillary comprising a tip orifice and an internal cavity, said internal cavity comprising a cell lysis buffer,
a positioning element capable of positioning the tip orifice of the capillary,
a discharging/drawing element capable of causing a discharge of liquid from the capillary as well as causing a drawing of liquid into the capillary,
said method comprising the steps of:

a. locating on the substrate the target cell to be isolated and lysed,

b. positioning the tip orifice of the capillary such that the tip orifice of the capillary comes in proximity to the target cell, using the positioning element,

c. discharging a sufficient volume of cell lysis buffer, preferably from the internal cavity of the capillary, such as to essentially enclose the target cell in the cell lysis buffer, using the discharging/drawing element

d. drawing the target cell enclosed in the cell lysis buffer into the internal cavity of the capillary, using the discharging/drawing element;

e. allowing the target cell to be lysed within the internal cavity of the capillary comprising the cell lysis buffer, and optionally

f. introducing a neutralising liquid into the internal cavity of the capillary such as to neutralise the cell lysis buffer or discharging the target cell lysate into a recipient preferably comprising a neutralising liquid such as to neutralise the cell lysis buffer.

It is further an object of the present invention to provide a method of isolating and proteolytically treating a target cell, preferably located on a substrate, using an apparatus comprising
at least a capillary comprising a tip orifice and an internal cavity, said internal cavity comprising a proteolytic buffer,
a positioning element capable of positioning the tip orifice of the capillary,
a discharging/drawing element capable of causing a discharge of the proteolytic buffer from the capillary as well as causing a drawing of the proteolytic buffer into the capillary,
said method comprising the steps of:

a. locating on the substrate a target cell to be isolated and proteolytically treated,

b. positioning the tip orifice of the capillary such that the tip orifice of the capillary comes in proximity to the target cell, using the positioning element,

c. discharging a sufficient volume of proteolytic buffer, preferably from the internal cavity of the capillary, such as to essentially enclose the target cell in the proteolytic buffer, using the discharging/drawing element

d. drawing the target cell enclosed in the proteolytic buffer into the internal cavity of the capillary, using the discharging/drawing element

e. allowing the target cell to be proteolytically treated within the internal cavity of the capillary comprising the proteolytic buffer, and optionally

f. introducing a neutralising liquid into the internal cavity of the capillary such as to neutralise the proteolytic buffer or discharging the proteolytically treated target cell into a recipient preferably comprising a neutralising liquid such as to neutralise the proteolytic buffer.

It is further an object of the present invention to provide an apparatus for use in a method of isolating and treating a particulate target, preferably located on a substrate, as described above, said apparatus comprising

a. at least a capillary comprising a tip orifice and an internal cavity configured to comprise a treatment liquid,

b. a positioning element configured to enable the positioning of the tip orifice of the capillary in proximity to the particulate target,

c. a discharging/drawing element configured to cause a discharge of treatment liquid from the capillary as well as causing a drawing of treatment liquid into the capillary when the positioning element.

It is further an object of the present invention to provide an apparatus for use in a method of isolating and lysing a cell, preferably located on a substrate, said apparatus comprising

a. at least a capillary comprising a tip orifice and an internal cavity configured to comprise a cell lysis buffer,

b. a positioning element configured to enable the positioning of the tip orifice of the capillary in proximity to the cell,

c. a discharging/drawing element configured to cause a discharge of the cell lysis buffer from the capillary as well as causing a drawing of cell lysis buffer into the capillary.

It is further an object of the present invention to provide an apparatus for use in a method of isolating and proteolytically treating a cell, preferably located on a substrate, said apparatus comprising

a. at least a capillary comprising a tip orifice and an internal cavity configured to comprise a proteolytic buffer,

b. a positioning element configured to enable the positioning of the tip orifice of the capillary in proximity to the cell,

c. a discharging/drawing element configured to cause a discharge of the proteolytic buffer from the capillary as well as causing a drawing of proteolytic buffer into the capillary.

Further embodiments are laid down in the claims as well as in the below detailed description of the invention.

### DESCRIPTION OF DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a capillary (3) having an internal cavity (4), and whose tip orifice is positioned in proximity of a particulate target (2) located on a substrate (1).

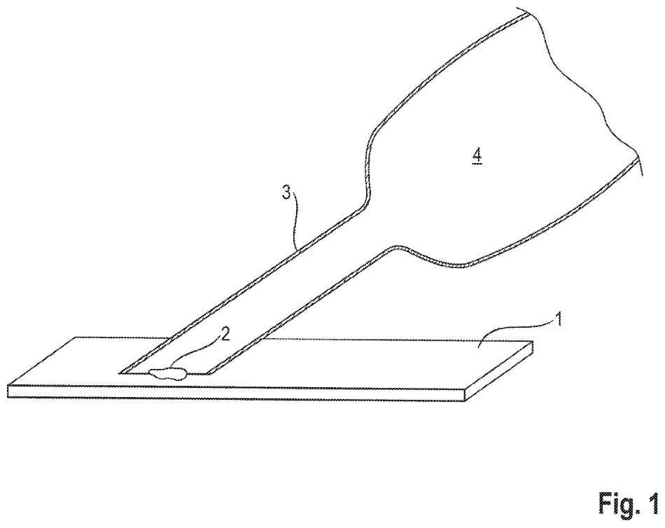

### DESCRIPTION OF PREFERRED EMBODIMENTS

It is thus an object of the present invention to provide a method of isolating and treating a particulate target, in particular a single particulate target, located using an apparatus comprising
at least a capillary comprising a tip orifice and an internal cavity, said internal cavity comprising a treatment liquid,
a positioning element capable of positioning the tip orifice of the capillary,
a discharging/drawing element capable of causing a discharge of liquid from the capillary as well as causing a drawing of liquid into the capillary,
said method comprising the steps of:

a. locating the particulate target to be isolated and treated;

b. positioning the tip orifice of the capillary such that the tip orifice of the capillary comes in proximity to the particulate target using the positioning element;

c. discharging a volume of treatment liquid such as to enclose the particulate target in the treatment liquid using the discharging/drawing element;

d. drawing the particulate target enclosed in the treatment liquid into the internal cavity of the capillary,

e. treating the particulate target within the internal cavity of the capillary comprising the treatment liquid and optionally

f. introducing a neutralising liquid into the internal cavity of the capillary such as to neutralise the treatment liquid or discharging the particulate target into a recipient preferably comprising a neutralising liquid such as to neutralise the treatment liquid.

In a preferred embodiment of the methods and apparatuses according to the present invention, the particulate target may be an inanimate particulate target particle, in particular a single inanimate particulate target particle, such as for example inorganic particulate targets or organic particulate targets, for example to be isolated and treated in the context of forensic investigations or geological investigations. Examples of such inanimate particulate targets are dust particles, crystal particles, diamond particles, salt particles, sulphide particles, oxide particles, and metal particles. In particular, the inanimate particulate targets may be embedded in a natural or man-made material. Examples of such embedding are target particles of geological interest embedded in natural rock or target particles of forensic interest embedded in a paraffin matrix for conservation purposes.

In a preferred embodiment of the methods and apparatuses according to the present invention, the particulate target may further be a cell, in particular a single cell or an aggregate thereof, i.e. an either eukaryotic or prokaryotic single cell, or an aggregate thereof. An example of an aggregate of prokaryotic cells is a colony of bacterial or fungal cells, whereas an aggregate of prokaryotic cells can be a tissue sample or aggregated cells from a cell culture. In particular the particulate target may also be a single animal cell or an aggregate thereof such as circulating cells like a leukocyte, thrombocyte or erythrocytes, or cultured single animal cells such as non-adherent cells and adherent cells. Single animal cells which are of particular interest in the diagnostic context are circulating tumour cells (CTCs) which may be single CTCs or CTC clusters, which can for example be obtained by filtration from blood samples. In order to case isolation of the cell, it may be labelled for example using fluorescent labels or dyes as is known in the art.

In a preferred embodiment of the methods and apparatuses according to the present invention, the particulate target is a cell such as a eukaryotic or prokaryotic cell, or an aggregate thereof, and preferably is an single animal cell or an aggregate thereof.

In a preferred embodiment of the methods and apparatuses according to the present invention, the treatment liquid is not a growth medium. The treatment liquid may be a enzymatic buffer for a proteolytic treatment which can be used to dissociate aggregates of cultured animal cells or single cultured animal cells from the substrate on which they are being cultured. An example of such an enzymatic buffer is trypsin buffer.

In a preferred embodiment of the methods and apparatuses according to the present invention, the treatment liquid is not a growth medium. The treatment liquid is a cell lysis buffer or a cell dissociation buffer, for example when the particulate target is a cell which is to be genetically analysed. An example of such a cell lysis buffers are sodium dihydrogen phosphate/disodium hydrogen phosphate buffer, Tris-HCl buffer, and HEPES-NaOH buffer. In general, treating the particulate target, i.e. the cell, in the treatment liquid, i.e. in the lysis buffer, lasts of from 1 second to 10 minutes, preferably of from 5 seconds to 5 minutes.

In a preferred embodiment of the methods and apparatuses according to the present invention, the particulate target, which is preferably an inanimate particulate target such as for example inorganic particulate target or organic particulate target is embedded in a solid material and the solid material is preferably soluble in the treatment liquid. The solid material can be a natural or man-made material. For example the treatment liquid may be either an organic solvent or an organic or inorganic acid.

In a preferred embodiment of the methods and apparatuses according to the present invention, the substrate may be any suitable body capable of carrying the particulate target, such as for example a glass slide, a polymer membrane, a filter plate or filter membrane, or the bottom plate of a cell culture container. In the case where the particulate target is an animal cell, and in particular where the animal cell is a circulating tumor cell (CTC) the substrate can be a filter plate or filter membrane.

In a preferred embodiment of the methods and apparatuses according to the present invention, the tip orifice of the capillary is positioned such that the orifice of the capillary comes at least within 20 μm, preferably within 10 μm, of the particulate target and/or wherein the particulate target has a size of from 100 nm to 100 μm, preferably of from 500 nm to 50 μm, more preferably of from 1 μm to 50 μm.

The apparatus that is used in the method of isolating and treating a particulate target according to the present invention comprises at least a capillary comprising a tip orifice and an internal cavity, said internal cavity comprising a treatment liquid, a positioning element capable of positioning the tip orifice of the capillary, a discharging/drawing element capable of causing a discharge of treatment liquid from the capillary as well as causing a drawing of treatment liquid into the capillary.

Capillaries comprising a tip orifice and an internal cavity can be purchased commercially. While the diameter of the tip orifice is not specially limited since it must be chosen such that the particulate target can enter the capillary, in most cases the diameter of the tip orifice will be in the range of 1 μm to 1000 μm, more preferably of from 10 μm to 100 μm and even more preferably of from 10 m to 50 μm. The diameter of the internal cavity is not specially limited, as long as the diameter of the internal cavity is at least equal to or larger than the diameter of the tip orifice. As an example a suitable diameter will be in the range of 100 μm to 5000 μm and preferably in the range of 100 μm to 1000 μm. The capillary may be formed from any suitable material such as glass, polymer or quartz.

In a preferred embodiment of the methods and apparatuses according to the present invention, the capillary can further comprise one or more integrated monitoring area configured to allow monitoring of the treatment of a particulate target in the internal cavity of the capillary by monitoring means such as for example optical means such as a spectrometer. The integrated monitoring area corresponds to an area on the capillary which area is configured to allow the transmission of electromagnetic radiation having a predetermined wavelength or range of wavelengths, such as for example UV, IR or VIS radiation, across the integrated monitoring area and into the internal cavity and back. Preferably, the integrated monitoring area is configured to allow the transmission of preferably at least 75% or at least 80% of a radiation. The integrated monitoring area may be made from different materials, depending on the required wavelength of the electromagnetic radiation used. For instance, suitable materials are glass having an optical wavelength transmission ranging of from 340 to 2,500 nm, polymer having a wavelength transmission range of from 380 to 780 nm (VIS) and quartz having a wavelength transmission range of below 380 nm (UV). The one or more integrated monitoring area can be formed for example as two diametrally opposed integrated monitoring areas or as one continuous integrated monitoring area encircling the capillary thus forming a ring integrated monitoring area.

In a preferred embodiment of the methods and apparatuses according to the present invention, drawing the particulate target enclosed in the treatment liquid into the internal cavity of the capillary is performed essentially immediately after enclosing the particulate target in the treatment liquid such that the treatment is essentially carried out in the internal cavity of the capillary. In particular in the case where the particulate target is a single cell or aggregate thereof, the drawing of the particulate target enclosed in the treatment liquid into the internal cavity of the capillary is preferably performed within seconds and more preferably within a minute after enclosing the particulate target in the treatment liquid.

In a preferred embodiment of the methods and apparatuses according to the present invention, the apparatus can further comprise a monitoring element configured to monitor the treatment of a particulate target in the internal cavity of the capillary, preferably via the integrated monitoring area. The monitoring element preferably comprises a radiation source and a radiation detector. The radiation source may be a light source capable of emitting UV, IR or VIS light within defined range of wavelength whereas the radiation detector is capable of either detecting light within the same defined range of wavelength or for example, as is the case when the treatment of the particulate target in the internal cavity of the capillary is monitored by measuring fluorescence, detecting light of a corresponding range of emitted wavelength. In a preferred embodiment of the methods and apparatuses according to the present invention, the radiation source, the integrated monitoring area and the radiation detector are arranged along a common axis.

It is thus a further object of the present invention to provide a method of isolating and treating a particulate target, in particular a single particulate target, such as a cell using an apparatus comprising

at least a capillary comprising a tip orifice and an internal cavity, said internal cavity comprising a treatment liquid, and an integrated monitoring area,

a positioning element capable of positioning the tip orifice of the capillary,

a discharging/drawing element capable of causing a discharge of liquid from the capillary as well as causing a drawing of liquid into the capillary,

a monitoring element capable of monitoring the treatment of a particulate target in the internal cavity of the capillary via the integrated monitoring area of the capillary,

said method comprising the steps of:

a. locating the particulate target to be isolated and treated;

b. positioning the tip orifice of the capillary such that the tip orifice of the capillary comes in proximity to the particulate target using the positioning element;

c. discharging a volume of treatment liquid such as to enclose the particulate target in the treatment liquid using the discharging/drawing element;

d. drawing the particulate target enclosed in the treatment liquid into the internal cavity of the capillary,

e. treating the particulate target within the internal cavity of the capillary comprising the treatment liquid,

f. determining if the treatment of the particulate target in the internal cavity of the capillary has completed using the monitoring element, and if so, optionally either

f. introducing a neutralising liquid into the internal cavity of the capillary such as to neutralise the treatment liquid or discharging the particulate target into a recipient preferably comprising a neutralising liquid such as to neutralise the treatment liquid.

In a preferred embodiment of the methods and apparatuses according to the present invention, the apparatus can further comprise a positioning element capable of positioning the tip orifice of the capillary which can either be operated manually or automatically. The capillary is in general fastened to the positioning element by way of a clamping element that clamps around a fastening section of the capillary. In one embodiment, the positioning element is a high precision robotic arm configured to position the tip orifice of the capillary in three dimensional space.

In a preferred embodiment of the methods and apparatuses according to the present invention, the apparatus can further comprise a discharging/drawing element capable of causing a discharge of treatment liquid from the capillary as well as causing a drawing of treatment liquid into the capillary can be a high precision pump that allows to either discharge or draw small volumes of treatment solution in the nanoliter range to take up single particulate targets such as animal cells for treatment. In general, the volume that is discharged and/or drawn can be selected in the range of 1 to 1000 nl, preferably in the range of 10 to 500 nl. The discharging/drawing element can either be operated manually or automatically. In a preferred embodiment, the particulate target enclosed in a volume of treatment liquid is drawn into the internal cavity of the capillary by preferably drawing of the same volume, or preferably lesser volume, of treatment liquid previously discharged to enclose the particulate target.

In a preferred embodiment of the methods and apparatuses according to the present invention and in the case where the capillary further comprises an integrated monitoring area configured to allow monitoring of the treatment of a particulate target in the internal cavity of the capillary by optical means, the apparatus further comprises a control element configured to i) receive information indicative of the progress of the treatment of the particulate target in the internal cavity of the capillary from the monitoring element and to ii) bring about the discharge of the particulate target from the capillary when receiving information indicative of the completion of the treatment from the monitoring element by controlling the discharging/drawing element.

In a preferred embodiment of the methods and apparatuses according to the present invention and in the case for example where the monitoring element comprises a radiation source and a radiation detector, the control unit is configured i) to receive information indicative of the progress of the treatment of the particulate target such as a cell in the internal cavity of the capillary in form of electrical or optical signals from both the radiation source and the radiation detector and, and ii) bring about the discharge of the particulate target such as a cell from the capillary receiving information indicative of the completion of the treatment from the monitoring element by controlling the discharging/drawing element.

In a preferred embodiment of the methods and apparatuses according to the present invention, the apparatus further comprises a releasing element configured to release the particulate target from a substrate by for example ultrasonic pulse or a laser pulse. In the case where the releasing element facilitates the release of the particulate target by ultrasonic pulse, the releasing element may be an ultrasonic transducer needle or spatula, configured to be positioned in proximity to the particulate target via for example a second positioning element. In the case where the releasing element facilitates the release of the particulate target by laser pulse, the releasing element may be a laser emitting element that can be focussed in proximity to the particulate target via for example a second positioning element and which can deliver consecutive pulses of 0.1-1000 μJ, preferably of 1-100 μJ and/or for about 1-100000 psec, preferably for about 1-10000 psec. The wavelength is preferably selected so that the particulate target is unaffected in its integrity, i.e. for the case of a cell is not killed. In one embodiment of the present invention, the releasing element may be a needle or spatula, without ultrasound or laser, formed from a suitable material such as polymer or metal.

In a preferred embodiment of the methods and apparatuses according to the present invention, at least the tip orifice of the capillary and/or the internal cavity and/or the releasing element is further lined with a hydrophobic layer such as for example a polysiloxan layer or fluoropolymer layer or with a hydrophilic layer to enhance the wetting of the tip orifice of the capillary and/or the internal cavity and/or the releasing element.

In a preferred embodiment of the methods and apparatuses according to the present invention, the tip portion and/or the longitudinal axis of the capillary is oriented in a normal direction with respect to a plane defined by the substrate or the tip portion and/or the longitudinal axis of the capillary is oriented in a non-normal direction with respect to a plane defined by the substrate and is preferably oriented of from 30 to 60 degrees with respect to the plane defined by the substrate.

LIST OF REFERENCE SIGNS

| | | | | |
|---|---|---|---|---|
| 1 | support | 5 | 3 | capillary |
| 2 | particulate target | | 4 | internal cavity |

1. A method of isolating and treating a particulate target using an apparatus comprising
at least a capillary comprising a tip orifice and an internal cavity, said internal cavity comprising a treatment liquid,
a positioning element capable of positioning the tip orifice of the capillary,
a discharging/drawing element capable of causing a discharge of treatment liquid from the capillary as well as causing a drawing of treatment liquid into the capillary, said method comprising the steps of:
a. locating the particulate target to be isolated and treated
b. positioning the tip orifice of the capillary such that the tip orifice of the capillary comes in proximity to the particulate target, using the positioning element,
c. discharging of treatment liquid such as to enclose the particulate target in the treatment liquid,
d. drawing the particulate target enclosed in the treatment liquid into the internal cavity of the capillary,
e. treating the particulate target within the internal cavity of the capillary comprising the treatment liquid, characterized in that the particulate target is a single animal cell or an aggregate thereof.

2. The method of isolating and treating a cell according to claim 1, wherein the treatment liquid is not a growth medium.

3. The method of isolating and treating a particulate target according to claim 1, wherein the treatment liquid is a cell lysis buffer or a proteolytic treatment buffer.

4. The method of isolating and treating a particulate target according to claim 1, wherein the orifice of the capillary is positioned such that the orifice of the capillary comes at least within 20 μm, preferably at least within 10 μm, of the particulate target and/or wherein the particulate target has a size of from 100 nm to 100 μm, preferably of from 500 nm to 50 μm and/or wherein the diameter of the tip orifice is in the range of 1 μm to 1000 μm, preferably of from 10 μm to 100 μm.

5. The method of isolating and treating a particulate target according to claim 1, wherein the particulate target substrate is located on a substrate preferably chosen from microscope slides, filter membranes such as CTC-filters or the bottom plate of a cell culturing container preferably containing cell culturing medium.

6. A method of isolating and lysing a target cell using an apparatus comprising
at least a capillary comprising a tip orifice and an internal cavity, said internal cavity comprising a cell lysis buffer,
a positioning element capable of positioning the tip orifice of the capillary,
a discharging/drawing element capable of causing a discharge of liquid from the capillary as well as causing a drawing of liquid into the capillary,
said method comprising the steps of:
a. locating the target cell to be isolated and lysed,
b. positioning the tip orifice of the capillary such that the tip orifice of the capillary comes in proximity to the target cell, using the positioning element,
c. discharging of cell lysis buffer such as to enclose the target cell in the cell lysis buffer, using the discharging/drawing element
d. drawing the target cell enclosed in the cell lysis buffer into the internal cavity of the capillary, using the discharging/drawing element;
e. allowing the target cell to be lysed within the internal cavity of the capillary comprising the cell lysis buffer, and optionally
f. introducing a neutralising liquid into the internal cavity of the capillary such as to neutralise the cell lysis buffer or discharging the target cell lysate into a recipient preferably comprising a neutralising liquid such as to neutralise the cell lysis buffer, characterized in that the target cell is a single animal cell or an aggregate thereof.

7. A method of isolating and proteolytically treating a target cell using an apparatus comprising
at least a capillary comprising a tip orifice and an internal cavity, said internal cavity comprising a proteolytic buffer,
a positioning element capable of positioning the tip orifice of the capillary,
a discharging/drawing element capable of causing a discharge of the proteolytic buffer from the capillary as well as causing a drawing of the proteolytic buffer into the capillary,
said method comprising the steps of:
a. locating a target cell to be isolated and proteolytically treated,
b. positioning the tip orifice of the capillary such that the tip orifice of the capillary comes in proximity to the target cell, using the positioning element,
c. discharging of proteolytic buffer such as to essentially enclose the target cell in the proteolytic buffer, using the discharging/drawing element
d. drawing the target cell enclosed in the proteolytic buffer into the internal cavity of the capillary, using the discharging/drawing element
e. allowing the target cell to be proteolytically treated within the internal cavity of the capillary comprising the proteolytic buffer, and optionally
f. introducing a neutralising liquid into the internal cavity of the capillary such as to neutralise the proteolytic buffer or discharging the proteolytically treated target cell into a recipient preferably comprising a neutralising liquid such as to neutralise the proteolytic buffer characterized in that the target cell is a single animal cell or an aggregate thereof.

8. An apparatus for use in a method of isolating and treating a particulate target according to claim 1, said apparatus comprising
a. at least a capillary comprising a tip orifice and an internal cavity configured to hold a treatment liquid,
b. a positioning element configured to enable the positioning of the tip orifice of the capillary in proximity to the particulate target,
c. a discharging/drawing element configured to cause a discharge of treatment liquid from the capillary as well as causing a drawing of treatment liquid into the capillary, characterized in that the particulate target is a single animal cell or an aggregate thereof.

9. The apparatus according to claim 8, wherein the capillary further comprises an integrated monitoring area to allow monitoring of the treatment of a particulate target in the internal cavity of the capillary and/or wherein the apparatus further comprises a monitoring element configured to monitor the treatment of a particulate target in the internal cavity of the capillary, preferably via said integrated monitoring area.

10. The apparatus according to claim 9, wherein it further comprises a control element configured to i) receive information indicative of the progress of the treatment of the particulate target in the internal cavity of the capillary from the monitoring element and to ii) bring about the discharge of the particulate target from the capillary when receiving information indicative of the completion of the treatment from the monitoring element by controlling the discharging/drawing element.

11. The apparatus according to claim 8, wherein it further comprises a releasing element configured to release the particulate target from a substrate by for example ultrasonic pulse or a laser pulse or mechanical action.

12. The apparatus according to claim 8, wherein at least the tip orifice of the capillary is further coated with a hydrophobic layer such as a polysiloxan layer and/or a hydrophilic layer.

13. An apparatus for use in a method of isolating and treating a particulate target according to claim 6, said apparatus comprising
a. at least a capillary comprising a tip orifice and an internal cavity configured to hold a cell lysis buffer,
b. a positioning element configured to enable the positioning of the tip orifice of the capillary in proximity to the particulate target,
c. a discharging/drawing element configured to cause a discharge of cell lysis buffer from the capillary as well as causing a drawing of cell lysis buffer into the capillary, characterized in that the particulate target is a single animal cell or an aggregate thereof.

14. The apparatus according to claim 13, wherein the capillary further comprises an integrated monitoring area to allow monitoring of the treatment of a particulate target in the internal cavity of the capillary and/or wherein the apparatus further comprises a monitoring element configured to monitor the treatment of a particulate target in the internal cavity of the capillary, preferably via said integrated monitoring area.

15. The apparatus according to claim 14, wherein it further comprises a control element configured to i) receive information indicative of the progress of the treatment of the particulate target in the internal cavity of the capillary from the monitoring element and to ii) bring about the discharge of the particulate target from the capillary when receiving information indicative of the completion of the treatment from the monitoring element by controlling the discharging/drawing element.

16. The apparatus according to claim 13, wherein it further comprises a releasing element configured to release the particulate target from a substrate by for example ultrasonic pulse or a laser pulse or mechanical action.

17. An apparatus for use in a method of isolating and treating a particulate target according to claim 7, said apparatus comprising
a. at least a capillary comprising a tip orifice and an internal cavity configured to hold a proteolytic buffer,
b. a positioning element configured to enable the positioning of the tip orifice of the capillary in proximity to the particulate target,
c. a discharging/drawing element configured to cause a discharge of proteolytic buffer from the capillary as well as causing a drawing of proteolytic buffer into the capillary, characterized in that the particulate target is a single animal cell or an aggregate thereof.

18. The apparatus according to claim 17, wherein the capillary further comprises an integrated monitoring area to allow monitoring of the treatment of a particulate target in the internal cavity of the capillary and/or wherein the apparatus further comprises a monitoring element configured to monitor the treatment of a particulate target in the internal cavity of the capillary, preferably via said integrated monitoring area.

19. The apparatus according to claim 18, wherein it further comprises a control element configured to i) receive information indicative of the progress of the treatment of the particulate target in the internal cavity of the capillary from the monitoring element and to ii) bring about the discharge of the particulate target from the capillary when receiving information indicative of the completion of the treatment from the monitoring element by controlling the discharging/drawing element.

20. The apparatus according to claim 17, wherein it further comprises a releasing element configured to release the particulate target from a substrate by for example ultrasonic pulse or a laser pulse or mechanical action.
